(19) United States
(12) Patent Application Publication
(10) Pub. No.: US 2014/0275894 A1
(43) Pub. Date: Sep. 18, 2014

(54) REFLUX PROBE

(71) Applicant: BABIESFIRST LIMITED, Hampton, Middlesex (GB)

(72) Inventor: Simon Bignall, Hampton (GB)

(73) Assignee: BABIESFIRST LIMITED, Hampton, Middlesex (GB)

(21) Appl. No.: 14/349,036

(22) PCT Filed: Oct. 3, 2012
(86) PCT No.: PCT/EP2012/069552

§ 371 Date: Apr. 1, 2014

(30) Foreign Application Priority Data
Oct. 3, 2011 (GB) 1117149.3

(51) Int. Cl. A61B 5/00

(52) U.S. Cl. CPC A61B 5/4211; A61B 5/0084
USPC 600/343; 600/309; 600/310

(57) ABSTRACT

A probe for detecting reflux in the oesophagus by detecting the level of turbidity at different points in the oesophagus. The probe consists of multiple pairs (32) of optical fibres with each pair being made up of a source optical fibre (2) and a detection optical fibre (3), a light source being attached to the source optical fibres and a light detector being attached to the detection optical fibres.

### TECHNICAL FIELD

This invention relates to probes for detecting reflux from the stomach to the oesophagus, and more specifically probes using electromagnetic radiation or light for detecting reflux from the stomach to the oesophagus. The invention is particularly useful for monitoring gastro-oesophageal reflux in small babies and/or babies born before completion of the full normal gestation period (i.e. pre-term or premature babies).

### BACKGROUND OF THE INVENTION

Reflux of stomach contents up the oesophagus (gastro-oesophageal reflux or GOR) may occur at any age but occurs more frequently in the first months of life. It does not always lead to symptoms, but can lead to aspiration into the lungs resulting in pneumonia or death. Inflammation in the oesophagus (oesphagitis) may lead to pain and symptoms such as restlessness, crying and aversion to feeding. Weight loss may occur with vomiting and reflux in infancy and is sometimes part of an allergy to cow's milk protein in infant formula, or to breast milk. The likelihood of GOR is much higher in premature babies or in those with neurological problems. The prevalence of reflux-associated disease (GORD) in infants has been reported as 2-10%, but is very high in very small premature babies recovering from intensive care.

Current apparatus for testing for reflux use indwelling pH probes housed in a nasogastric catheter (see, for example, US 2003/0028088) to measure acidity in the oesophagus, or indwelling impedance probes which measure the impedance of the oesophagus (see, for example, U.S. Pat. No. 5,833,625).

Stomach contents are usually acidic and known pH probes can detect the presence of acidic material with a pH below 4. The known pH probes have a relatively slow response time (i.e. time it takes for the probe to produce a signal corresponding to the presence of acidic material with a pH below 4) which means that there is an unavoidable timelag between the contacting of the probe with acidic material and an output signal informing an operator or monitoring apparatus that acidic material is present. PH probes cannot therefore give an immediate picture of what is happening in the oesophagus.

Furthermore, patients suffering from reflux are likely to be taking antiacid and other medications which mean that any refluxate may not be sufficiently acidic when an incidence of reflux occurs. The known pH probe systems are therefore inaccurate and likely to miss incidences of reflux unless the patient has his or her medication stopped. In some instances when using the known pH probes it is necessary to stop medication for 24 hours so as to avoid the medication contaminating the results from the probe. Stopping medication is clearly not ideal.

Furthermore, GOR in premature babies is also difficult to identify by current pH probe methods as the continual or very frequent milk feeds provided to premature babies buffer the acidity needed for pH probes to work, and the slow response of such pH sensors mean that one cannot compensate for known feeding times as the timelag associated with the sensors means that it is not possible to accurately determine the timing of a reflux incident.

It has not been possible to make an impedance sensor which works effectively in a clinical environment. One of the problems with impedance is that the output signals have significant noise. The possible causes of this include the fact that muscles themselves produce currents, and the fact that the pathways in the oesophagus for the flow of current from and to a probe are complicated and difficult to model accurately.

GOR causes long stays in hospital as well as the need for intensive nursing and medical therapy. The problems associated with the current known reflux sensing arrangements for identification and monitoring of instances of gastro-oesophageal reflux, means that effective areal-time monitoring for reflux remains elusive to clinicians; particularly when looking after small infants and preterm babies.

There is therefore a need for an improved method and apparatus for diagnosing and monitoring gastro-oesophageal reflux, and in particular for diagnosing and monitoring gastro-oesophageal reflux in premature or small babies.

### SUMMARY OF THE INVENTION

The invention is defined in the appended independent claims to which reference may now be made. Advantageous features of the invention are set forth in the dependent claims.

The invention provides a reflux probe for insertion into an oesophagus and the monitoring of gastro-oesophageal reflux by monitoring the turbidity of the contents of an oesophagus. Turbidity is a measure of the concentration of particles suspended in a liquid; it can be thought of as the cloudiness of a suspension or emulsion.

The inventor is the first to appreciate that a problem with the existing pH and impedance probes arises from the fact that they do not directly monitor reflux. They monitor parameters or events (e.g. acidity and impedance levels) which are proxy indicators of the presence of reflux but which can be associated with events other than gastro-oesophageal reflux and/or which are not always present when gastro-oesophageal refluxate is present.

A different indicator of the presence of reflux is therefore desirable if one is to accurately monitor reflux in situations where milk, medication or some other fluid may be present and buffers the acidity of stomach contents. The inventor is the first to appreciate that one can use the fact that gastro-oesophageal reflux from a baby will be mainly milk and therefore an emulsion.

The inventor is the first to appreciate that once one recognises that the gastro-oesophageal refluxate from a baby or young infant is mainly milk and therefore an emulsion, it's presence or absence can be monitored by taking advantage of the fact that the presence of an emulsion (i.e. refluxate or milk) can be monitored or determined by monitoring the turbidity or cloudiness of the contents of the oesophagus.

The monitoring of turbidity allows the use of optical components which have a significantly faster response time than the know pH sensors, and are cheaper. The probe of the current invention therefore provides the advantages of reduced cost and real-time demonstration of the level to which reflux occurs, allowing the continuous, bedside display of the effects of anti-reflux medication, medical, surgical and nursing procedures on the extent of GER) i.e. gastro-oesophageal reflux).

In addition, the known pH and impedance sensors are expensive which limits the number of sensing locations that it is economic to provide in a single sensor. The known reflux sensors are limited to 4 sensing points, due to cost. There is no such limitation with the optical method of the current invention allowing more sensing points spaced closer together if required, a particular advantage in small babies with a short oesophagus where precise delineation of the level of reflux is key to therapeutic decision-making.

Preferably, the probe has a first proximal end for coupling to data processing apparatus outside a body, and a second distal end, the probe comprising: at least one pair of conduits for electromagnetic radiation, the pair of conduits comprising a source conduit and a detection conduit, and each conduit having an input for receiving electromagnetic radiation and an output for onward transmission of electromagnetic radiation, the source conduit being arranged for coupling at its input to an electromagnetic radiation source unit to provide electromagnetic radiation through the source conduit to a sensing location at or near the input of the detection conduit, the detection conduit being arranged for coupling at its proximal output end to an electromagnetic radiation detector unit so as to transmit electromagnetic radiation from the sensing location at its input, to the electromagnetic radiation detector unit.

This probe construction allows for a relatively robust and effective probe capable to monitoring turbidity.

Preferably the reflux probe includes at least two detection conduits, and wherein the inputs of the detection conduits are spaced from each other so that, in use, each detection conduit is arranged to transmit electromagnetic radiation from a different sensing location within the oesophagus, to the electromagnetic radiation detection unit.

This allows for the monitoring of reflux at different locations within the oesophagus. This is important for therapeutic reasons as it allows the determination of the level or extent of reflux. The fast response time of the optical sensing arrangements also mean its is possible using appropriate data processing of the signals produced by such a probe to determine the order in which an emulsion is detected at the different sensing locations and hence determine the direction in which the emulsion is moving through the oesophagus.

Preferably the reflux probe includes a separate source conduit corresponding to each detection conduit fibre, and wherein the output of each source conduit is adjacent or near to the input end of a respective detection conduit. This makes for an easily controllable probe in which is possible to accurately direct light or electromagnetic radiation from a particular source conduit into a particular selected detection conduit.

Preferably the reflux probe includes at least two pairs of adjacent source and detection conduits.

Preferably the inputs of the detection conduits are spaced relative to each other along a longitudinal axis of the probe. This allows for the sensing of the turbidity of the oesophagus at different points along the longitudinal axis of the probe. As the probe is likely to be inserted with its longitudinal axis parallel to the length of the oesophagus this allows for the determination of the turbidity at different depths of the oesophagus. This in turn allows for a determination of the extent or height of reflux, and the distinguishing between milk being fed into the oesophagus from the mouth, and refluxate rising up into the oesophagus form the stomach (i.e. in opposite direction to milk being fed to a patient).

Preferably the inputs of the detection conduits are approximately 1 cm apart along the longitudinal axis of the probe. This allows for effective sensing and therapeutically effective reflux data from a patient.

Preferably the output or outputs of the source conduit and the input of the respective detection conduit point away from each other. This reduces the risk that light from a source conduit impinges on the detection optical fibre directly, so that only diffuse light is collected.

Preferably the output end or ends of the source and corresponding input end of ends of the detection optical fibres are each arranged at approximately 45° to the longitudinal axis of the probe and each point away from each other. This angle ensures an appropriate level of diffuse light collection while still avoiding collection of direct light.

Preferably the source conduit or conduits and/or the detection conduit or conduits is or are an optical fibre or fibres. Optical fibres are a robust and cost-effective conduit for electromagnetic radiation of the appropriate wavelength (i.e. a wavelength which is scattered by particles of the size which are present and suspended in milk).

Preferably optical fibre or fibres have a first input end and second output end. Transmission of electromagnetic radiation or light through an optical fibre so that it exits from an output end allows for accurate and reliable supply of the radiation to a selected probe location.

In an alternative embodiment, the source conduit is an optical fibre which transmits light along its length and the probe includes a coloured overlay having a number of different colours along its length and arranged around the optical fibre so that, in use, the light entering the oesophagus has different wavelengths at different depths within the oesophagus. This allows one to use a single source conduit to supply radiation or light to a number of different locations and to differentiate between the radiation or light supplied to the different locations.

In an alternative embodiment the source conduit or conduits and/or the detection conduit or conduits is or are a catheter or catheters

Preferably the probe includes a light source at its proximal end and the input of the source conduit is at the probe's proximal end for onward transmission of light from the light source. This allows for relatively easy construction of the probe.

In an alternative embodiment, the probe includes a light source at its distal end and the input of the source conduit is at the probe's distal end for onward transmission of light from the light source.

Preferably the probe includes a light source which provides light of a wavelength or wavelengths which is scattered by particles present in milk.

Preferably the invention provides a reflux monitoring apparatus including a reflux probe and an electromagnetic radiation detection unit which monitors the wavelength and/or amplitude of the radiation transmitted by the output or outputs of the detection conduit or conduits.

Preferably the apparatus monitors or measures the nephelometric turbidity in the oesophagus. Nephelometry is the measurement of the size and concentration of particles in a liquid by analysis of light scattered by the liquid.

Preferably, the first ends of the source and detection optical fibres of each pair of optical fibres are arranged proximate one another. This allows a good level of incident light on the first end of the detection optical fibre

Preferably, the optical fibres of each pair of optical fibres are coupled to each other by biocompatible cladding or sheathing. This cladding or sheathing can protect and/or support the optical fibres.

Unlike the current methods, the optical or photometric methods possible with the subject invention do not rely on a change in ionic content. The methods therefore have a distinct advantage particularly in newborns where non-acidic reflux and delayed fluid clearance is prevalent. A further advantage over the measurement of intra-oesophageal pH is that an optical method will work in the presence of antacid drugs and therapies which prevent the detection of acidity as contents reflux into the oesophagus. When pH measurement is used, drugs must be stopped for 24 hours beforehand in order to retain gastric and oesophageal acidity.

An optical method will also allow very rapid determination of the depth of the refluxate and will not require the clinical skill in interpretation and the computation currently required for some methods of GOR measurement.

The probe and apparatus of the current invention and methods associated with such a probe or apparatus are uniquely capable of measuring reflux along the length of the oesophagus from stomach to pharynx. The fast response times allow a continuous bedside display of the anatomical level of reflux specific to the individual under study. This continuous display and measurement will allow the effects of treatments to be visible to nursing and medical staff at once. This method has an essential role in all hospitals for identifying reflux in babies in particular, for example with intensive or special care baby units and on paediatric units caring for babies in the first year of life.

### BRIEF DESCRIPTION OF THE DRAWINGS

An embodiment of the invention will now be described by way of non-limiting example and with reference to the accompanying drawings in which:

FIG. 1a is a cross-sectional side view of a probe embodying the invention, and figure lb illustrates a detail of an end of the probe of FIG. 1;

FIG. 2 is a graph showing the results of an in vitro experiment illustrating how the apparatus of claim 1 monitors the presence and absence of milk;

FIG. 3 is a cross-sectional side view of a probe in a second embodiment of the invention similar to that of FIG. 1 but in which there are a three pairs of optical fibres;

FIG. 4 is a cross-sectional side view of a probe in a third embodiment of the invention incorporating a single central lossy source fibre and four external detection fibres;

FIG. 5 is a cross-sectional side view of a probe in a fourth embodiment of the invention incorporating a single catheter which may receive refluxate in its interior, and in which light transmitted through the catheter (and any refluxate or other material contained therein) is reflected back through a channel on the outside of the catheter;

FIG. 6 is a cross-sectional side view of a probe in a fifth embodiment of the invention in which light is reflected from a distal mirror or reflector to a number of sensing points; and

FIG. 7 is a cross-sectional side view of a probe in a sixth embodiment of the invention with a light source at the distal end of the probe

### DETAILED DESCRIPTION OF THE PREFERRED EMBODIMENTS

A probe for detecting reflux in the oesophagus according to the invention will now be described. FIG. 1 shows part of a preferred embodiment, in which a first source optical fibre 2 and a second detection optical fibre 3 are held within a sheath or cladding 4 made of biocompatible material. At a first distal end 6 of the probe and the optical fibres 2, 3, the optical fibres 2, 3 are cut at 45 degrees to the longitudinal axis of the optical fibres 2, 3. The longitudinal axis of the probe runs along the length of the optical fibres 2, 3. The optical fibres are arranged such that the cut faces are angled away from each other. A second proximal end (not shown) of the source optical fibre 2 is attached to an electromagnetic (or light) source such as an. LED, and a second proximal end of the detection optical fibre 3 is attached to an electromagnetic radiation (or light) detector such as a photodiode receptor. The electromagnetic or light detector is coupled to a data processor which processes the data or information received by the detector so as to analyse the intensity and wavelength of the light which reaches the detector.

The turbidity or cloudiness of the contents of the oesophagus can be measured using a probe such as that shown in FIG. 1. In the example probe shown in FIG. 1, turbidimetry is used; that is, the light reflected off particles is monitored rather than the attenuation of light.

The probe is first inserted into the oesophagus of the patient, typically via the nasal cavity into the oesophagus, although it could be passed by another route.

Light is then passed through the source optical fibre 2 and any resultant scattered light passes up the detection optical fibre 3, and is then detected or collected by the light detector. The light collected is integrated and analysed over periods of, for example, 500 ms.

Sample results are shown in the graph in FIG. 2, which shows the effect of radial scattering of 100% milk in increasing the transmission and reception of red light by the detection optical fibre, an increase of fourfold from transmission though pure water. The wavelength of the light is shown against the intensity of the scattered light (shown as percentage saturation). A 660 nm light source was used. The solid line 101 shows the results when probe was placed in pure water (which represents the state of no reflux in the oesophagus) and the dotted line 100 shows the results when the probe was placed in milk (which represents the state of refluxate in the oesophagus). The intensity of scattered light in this example is approximately 4 times greater in the presence of milk than without. Increasing dilutions of milk were distinguishable from water until the proportion of milk was less than 1 in 20.

FIG. 3 shows a probe in a second preferred embodiment of the invention. The probe consists of three pairs of fibres 32. Each pair of fibres consists of two connected or coupled optical fibres, with the first distal ends 6 cut at 45 degrees as described above in connection with FIG. 1. In this example, the pairs of fibres 32 are arranged so that the first distal ends 6 of the three pairs of fibres are spaced 1 cm apart in the direction of the longitudinal axis of the fibres, although other spacings could also be used.

While three pairs of fibres are shown in this example, two, four, or more fibres can also be used. It is envisioned that embodiments of the invention could also consist of 10 to 100 pairs of fibres. Clinically a separation of 1 cm corresponding to measurements at 1 cm along the length of the oesophagus allow for therapeutically and diagnostically useful data. A small infant's oesophagus might be 10 cm long so 10 sensing locations separated by 1 cm would allow a thorough picture of the extent of refluxate in the entire oesophagus to be determined. The selection of the number of sensing locations, the length of the sensing position of the probe and the separation between sensing locations is determined by the size of the oesophagus being monitored and the clinical information desired by the patient's carers. This is a particular advantage in small babies with a short oesophagus where precise delineation of the level of reflux is key to therapeutic decision-making. The separation between sensing locations is a function of the number of sensing points and the length of the oesophagus being monitored.

An adult oesophagus is typically 25 to 30 cm so more sensing locations and/or a greater or different separation between sensing locations might be desirable.

In the embodiment shown in FIG. 4, the probe comprises a single central source fibre 7 and four detection or receiving fibres 3 arranged around the outside of the probe. The detection fibres each finish at a different location along the longitudinal axis of the probe so as to receive or detect light at a different depth when the probe is inserted into an oesophagus. The central fibre 7 is a fibre which transmits light outwards along its length. An overlay 8 of a coloured material is arranged around the central source fibre. The overlay has a different colour (i.e. filters light of different wavelength) at each location which corresponds to the input to each respective detection fibre 3 so that the light entering each detection fibre has a particular wavelength. This allows the light detector associated with the outputs from the detection fibres to differentiate between the light coming from different depths and hence different detectors. An alternative to the overlay would be the painting or different colours directly onto the central fibre.

Referring to FIG. 5, in an alternative embodiment of the invention the probe includes a single transparent plastic catheter 9 which has an opening so as to receive any fluid present in the oesophagus in its interior space when placed in the oesophagus. The probe includes a light source (not shown) which transmits light into the interior of the catheter from its proximal end. The catheter's exterior surface has an overlay or painted surface with different colours or light filters at different points along its length similar to that described above in connection with FIG. 4. A terminal reflective surface 10 is provided at the probe's distal end. Light transmitted outwards from the catheter at different points along its length is channelled to detection apparatus at the probe's proximal end or connected to the probe's proximal end via conduit 3 and monitored in a manner similar to that described above in connection with FIG. 4.

FIG. 6 shows another embodiment in which a single central fibre optical 40 transmits light from a light source (not shown) and terminates proximal to a mirror or reflective surface 42. In the illustrated embodiment two source optical fibres 21, 22 extend from proximate the mirror 42 to proximate a first input end of respective detection optical fibres 31. 33. The input ends of the detection optical fibres 31, 32 are arranged such that they overlap the outputs ends of the respective source optical fibre 21, 22 at respective measurement points 51, 52, whilst being adjacent the source fibre in the longitudinal axis. The measurement points 51, 52 are at different points along the probe so as to monitor turbidity at different depths in the oesophagus.

In FIG. 6, two pairs of source optical fibres 21, 22 and second optical fibres 31, 32 are shown, but one, three, or more pairs of fibres may be provided.

FIG. 7 shows another embodiment similar to that shown in FIG. 4. In this embodiment, a light source 50 is arranged at the distal end of the probe and the light detector (not shown) is arranged at the other proximal end of the probe. The light source 50 is arranged proximate the second end of two source optical fibres 2. At the measurement points 51, 52, the first ends of two detection optical fibres 31, 32 are arranged adjacent to the first ends of the two corresponding source optical fibres 21, 22, similarly to the source and detection optical fibres in FIG. 6. This provides two pairs of source and detection optical fibres. A light detector (not shown) is attached to the second end of the second optical fibres. The measurement points 51, 52 are spaced at longitudinally different points of the probe.

The embodiments described above show one, two or three pairs of optical fibres for simplicity. Four or more pairs of optical fibres may also be used, and in some embodiments a considerable number of pairs, for example 10 or 20 pairs, would be most preferable. In particular, an envisaged set of embodiments comprise probes of between 10 and 100 cm in length with an optical fibre pair terminating at centimetre intervals along the length of the probe.

While the various embodiments described all show the measurement points being spaced apart longitudinally along the probe for each separate fibre, two fibres measuring at the same longitudinal point can be provided. This can improve accuracy by providing two independent measurements at the same point in the oesophagus. Multiple or contiguous sites of recording of the presence of reflux at different positions along the probe allow a precise identification of the highest level to which reflux occurs within the oesophagus. In particular, this can help allow for the exclusion of artefacts in the signal related to the position of the probe next to the oesophageal wall, which might exclude the refluxate from the area of the local optical sensors and prevent the scattering of light which demonstrates the presence of refluxate.

Where multiple optical fibres are provided, they are preferably substantially parallel.

In the embodiments in FIGS. 1 and 3, the ends of the optical fibres are cut at an angle to ensure that transmitted light is beamed away from the receiving fibre terminations.

Other ways of beaming light exiting the source fibre(s) away from the detection fibres are also possible. These include fibre terminations including a reflective/refractive element, or optical fibres that are bent to point away from each other. Preferably all embodiments illustrated provide a mechanism for pointing light exiting the source fibre(s) away from the respective detection fibre(s) so as to try and ensure that only scattered light enters each detection fibre or conduit.

The example of FIG. 2 uses light at 660 nm, but other options are available. Any electromagnetic radiation having a wavelength which means that it is scattered or affected by particles of the size suspended in milk is suitable. White light with coloured filters could be used to allow identification of the level of reflux. Two-wavelength spectroscopy could also be used, with for example 660 and 900 nm light, to determine any interference by the haeomoglobin-containing mucosal surface of the oesophageal wall. Other wavelengths of electromagnetic radiation can also be used.

By the use of different wavelengths of light, and filters/dyes arranged along the array of sensors, the use of a white light used to interrogate the entire length of the probe can be converted into corresponding depth with an accuracy limited only by the number of filters/dyes of different wavelengths used and the accuracy of the spectrophotometric determination of the light reflected up the single or multiple optical fibres

The light source may be a laser, an LED, or another suitable light source. In embodiments where light must be entered into one more than one optical fibre, the light source can comprise a single source from which the light is split up for the separate fibres, or a plurality of sources.

The light detector may be a photodiode or an electromagnetic detection unit such as an array of diodes. One example is a digital spectrophotometer such as one available from Nicholl Education Ltd, UK with integration times of 500 ms. Other integration times may be used.

A variety of attachment techniques may be used to join the fibres together in the embodiments described, such as cladding, glue or ties or attachments spaced along the fibres periodically. FIG. 1 shows an example of cladding or a sheath and FIG. 3 shows an example of periodic attachments or ties 34. Multiple pairs of cables can be attached to one another in a similar manner. Cladding could take the form of some kind of sheath or of tape wound around the cables to bind them together.

Various modifications to the embodiments described are possible and will occur to those skilled in the art without departing from the invention which is defined by the following claims.

1. A reflux probe configured for insertion into an oesophagus and for monitoring of gastro-oesophageal reflux by monitoring turbidity of contents of the oesophagus.

2. The reflux probe according to claim 1, wherein the reflux probe has a first proximal end for coupling to a data processing apparatus outside a body, and a second distal end, the reflux probe comprising:
at least one pair of conduits configured for propagating electromagnetic radiation, the pair of conduits comprising a source conduit and a detection conduit, and each conduit having an input for receiving electromagnetic radiation and an output for onward transmission of electromagnetic radiation,
the source conduit being arranged for coupling at its input to an electromagnetic radiation source unit to provide electromagnetic radiation through the source conduit to a sensing location at or near the input of the detection conduit, the detection conduit being arranged for coupling at its proximal output end to an electromagnetic radiation detector unit so as to transmit electromagnetic radiation from the sensing location at its input to the electromagnetic radiation detector unit.

3. The reflux probe according to claim 2, wherein the reflux probe includes at least two detection conduits, and wherein the inputs of the detection conduits are spaced from each other so that, in use, each detection conduit is arranged to transmit electromagnetic radiation from a different sensing location within the oesophagus to the electromagnetic radiation detection unit.

4. The reflux probe according to claim 3, wherein the reflux probe includes a separate source conduit corresponding to each detection conduit, and wherein an output of each source conduit is adjacent or near to the input of a respective detection conduit.

5. The reflux probe according to claim 4, including at least two pairs of adjacent source and detection conduits.

6. The reflux probe according to claim 3, wherein the inputs of the detection conduits are spaced relative to each other along a longitudinal axis of the probe.

7. The reflux probe according to claim 6, wherein the inputs of the detection conduits are spaced approximately 1 cm apart along the longitudinal axis of the probe.

8. The reflux probe according to claim 2, wherein the output of the source conduit and the input of the respective detection conduit point away from each other.

9. The reflux probe according to claim 8, wherein the output end of the source conduit and corresponding input end of the detection conduit are each arranged at approximately 45° to the longitudinal axis of the probe and each point away from each other.

10. The reflux probe according to claim 2, wherein at least one of the source conduit and the detection conduit is an optical fibre.

11. The reflux probe according to claim 10, wherein the optical fibre has a first input end and second output end.

12. The reflux probe according to claim 2, in which the source conduit is an optical fibre which transmits light along its length and the reflux probe includes a colored overlay having a number of different colors along its length and arranged around the optical fibre so that, in use, the light entering the oesophagus has different wavelengths at different depths within the oesophagus.

13. The reflux probe according to claim 2, wherein at least one of the source conduit and the detection conduit is a catheter. catheters.

14. The reflux probe according to claim 2, wherein the probe includes a light source at its proximal end and the input of the source conduit is at the probe's proximal end for onward transmission of light from the light source.

15. The reflux probe according to claim 2, wherein the probe includes a light source at its distal end and the input of the source conduit is at the probe's distal end for onward transmission of light from the light source.

16. The reflux probe according to claim 2, wherein the reflux probe includes a light source which provides light of wavelengths that are scattered by particles present in milk.

17. A reflux monitoring apparatus including the reflux probe according to claim 2, and further including an electromagnetic radiation detection unit which monitors at least one of wavelength and amplitude of radiation transmitted by the output of the detection conduit.

18. The reflux monitoring apparatus according to claim 17, configured for measuring nephelometric turbidity in the oesophagus.

19. A method for measuring oesophageal turbidity comprising:
providing a reflux probe configured for insertion into an oesophagus and configured for monitoring of gastro-oesophageal reflux by monitoring turbidity of contents of the oesophagus, the reflux probe having a first proximal end for coupling to a data processing apparatus outside a body and a second distal end;
providing the reflux probe with at least one pair of conduits configured for propagating electromagnetic radiation, the pair of conduits comprising a source conduit and a detection conduit, each conduit having an input for receiving electromagnetic radiation and an output for onward transmission of electromagnetic radiation, the source conduit being arranged for coupling at its input to an electromagnetic radiation source unit to provide electromagnetic radiation through the source conduit to a sensing location at or near the input of the detection conduit, the detection conduit being arranged for coupling at its proximal output end to an electromagnetic radiation detector unit so as to transmit electromagnetic radiation from the sensing location at its input to the electromagnetic radiation detector unit.

20. The method according to claim 19, further comprising monitoring gastro-oesophageal reflux by monitoring the turbidity of the contents of an oesophagus with the reflux probe.

21. The method according to claim 20, wherein the turbidity of the contents of an oesophagus is monitored at different depths within the oesophagus.

22.-24. (canceled)
